(19) United States
(12) Patent Application Publication
(10) Pub. No.: US 2021/0209271 A1
(43) Pub. Date: Jul. 8, 2021

(54) FLEXIBLE DISCRETE EVENT SIMULATOR

(71) Applicant: Battelle Memorial Institute, Columbus, OH (US)

(72) Inventors: Jarrod R. Olson, Columbus, OH (US); Celeste Vallejo, Columbus, OH (US); Po-Hsu Chen, Columbus, OH (US); Tyler Coy, Columbus, OH (US)

(21) Appl. No.: 17/143,244

(22) Filed: Jan. 7, 2021

Related U.S. Application Data
(60) Provisional application No. 62/959,390, filed on Jan. 10, 2020.
(60) Provisional application No. 62/957,943, filed on Jan. 7, 2020.

(51) Int. Cl. G06F 30/20; G16H 10/60; G16H 50/70; G06Q 10/06; G06Q 40/08; G06F 9/54

(52) U.S. Cl. CPC G06F 30/20; G16H 10/60; G16H 50/70; G06F 2111/08; G06Q 40/08; G06F 9/541; G06Q 10/067

(57) ABSTRACT

In discrete event simulation (DES), a timepoint for the DES is incremented. At the increments, event modules are executed to process members of at least one population class according to flow of the members amongst the event modules as defined by application programming interfaces (APIs). The event modules transform attributes of the members according to probabilistic event models defined by event model parameter files. DES data comprising attributes of the members at end-of-simulation are stored. In another aspect, simulation of a system model having parameters with associated probability density functions (PDFs) includes M outer loops each including: for each parameter, randomly drawing a parameter value with replacement from a set of discrete sampling points distributed over the PDF of the parameter; and running N simulations of the system using the system model with the randomly drawn parameter values and storing the simulation results annotated by the randomly drawn parameter values.

### CROSS-REFERENCE TO RELATED APPLICATIONS

This application claims the benefit of U.S. Provisional Application No. 62/957,943 filed Jan. 7, 2020 and titled “FLEXIBLE DISCRETE EVENT SIMULATOR”. This application also claims the benefit of U.S. Provisional Application No. 62/959,390 filed Jan. 10, 2020 and titled “SIMULATION WITH DYNAMIC MONTE CARLO SAMPLING”.

U.S. Provisional Application No. 62/957,943 filed Jan. 7, 2020 and titled “FLEXIBLE DISCRETE EVENT SIMULATOR” is incorporated herein by reference in its entirety. U.S. Provisional Application No. 62/959,390 filed Jan. 10, 2020 and titled “SIMULATION WITH DYNAMIC MONTE CARLO SAMPLING” is incorporated herein by reference in its entirety.

### BACKGROUND

The present disclosure relates generally to the discrete event simulation (DES) arts, and to applications of DES, and to model simulation arts, simulation statistical analysis arts, simulation reliability assessment arts, and the like.

Simulation of a real-world system is a powerful tool for studying the system and predicting the effects of possible modifications to the system. By way of a few nonlimiting illustrative examples, systems amenable to simulation include roadway traffic systems (e.g. roadway networks, trucking fleets), aviation networks (e.g., airports, airline hub networks), computer networks (e.g. modeling data flow to study possible bottlenecks or the like), manufacturing facilities (e.g. modeling assembly lines), medical services (e.g. services delivery and reimbursement), financial systems (e.g. stock markets), and so forth.

In a typical approach, a simulation models the system using a probabilistic model having a set of random variables and a set of parameters. The random variables represent events that may occur with certain likelihoods and may have various possible outcomes with certain likelihoods, and attributes of members of a population interacting with the system. For example, in modeling a trucking fleet, random variables may be used to represent likelihood of events such as a truck being involved in a roadway accident, a delay due to roadway congestion, or so forth; attributes of the trucks such as fuel efficiency, and attributes of truck drivers such as average number of driving hours per day for a given driver. Parameters of this illustrative trucking fleet model may, for example, include the number of trucks in the fleet, the number of drivers, and parameters of probability density functions (PDFs) that represent the random variables.

One commonly used simulation technique is discrete event simulation (DES), which is a methodology that allows its users to treat a series of events as probabilistic and link them together to model a system and potential emergent behavior. A core strength of DES is the ability to provide alternative event or attribute specifications to understand the uncertainty of the model, and to rapidly prototype alternative innovations. Alternative specifications of a certain type of event can be represented, for example, by using adjustable parameters in a PDF of a random variable representing the event type. For the illustrative trucking fleet model, as an example, the fuel efficiency of trucks of the fleet may be represented by a random variable having a Gaussian PDF. The mean of the Gaussian PDF represents the average fuel efficiency of all trucks in the fleet, while the variance of the Gaussian PDF represents the “spread” or “variation” of fuel efficiency amongst the trucks of the fleet. In this example, switching from a more expensive grade of diesel fuel that provides higher fuel efficiency to a less expensive grade of diesel fuel that provides lower fuel efficiency could be simulated by adjusting two or three model parameters: the price of diesel fuel, the mean of the Gaussian PDF of the random variable representing truck fuel efficiency, and possibly also the variance of the Gaussian PDF. A first set of simulations can then be run using the first set of parameters in order to model trucking fleet operations using the more expensive grade of diesel fuel; and likewise a second set of simulations can then be run using the second set of parameters in order to model trucking fleet operations using the less expensive grade of diesel fuel. By way of such simulations, it can be assessed whether switching to the less expensive grade of diesel fuel would result in an overall cost savings for the trucking fleet.

Because the simulation employs a probabilistic model of the system, multiple runs of the simulation are usually performed to generate sufficient statistics to assess the desired information. However, analysis of the generated statistics is complex. Uncertainty in model output can be classified into two categories: aleatoric uncertainty and epistemic uncertainty. Aleatoric uncertainty refers to the uncertainty driven by the stochasticity or randomness of the model (that is, the inherent randomness of the random variables of the model). For a given set of parameters, the amount of aleatoric uncertainty can be visualized from the variance of model output over multiple runs of the model. Increasing the number of model runs is a way to reduce aleatoric uncertainty in the results.

Epistemic uncertainty refers to the uncertainty in the values of the parameters of the model. Using the trucking fleet as a continuing example, the model parameter representing prices of the higher and lower grades of diesel fuel have uncertainty since the price of diesel varies over time based on economic market forces. The mean and variance parameters of the PDF representing fuel efficiency may also have some uncertainty. For example, if the fleet has been running on the more expensive grade of fuel, there may be no direct information on the fuel efficiency that would be obtained using the less expensive grade of fuel, and hence the values for the mean and variance parameters of the fuel efficiency PDF may need to be estimated based on less satisfactory sources (e.g. limited test runs with a few trucks using the less expensive grade of diesel, comparisons with other trucking fleets that use the less expensive grade of diesel but which may differ in other ways from the fleet under simulation, or even simply taking fuel efficiency ratings for the less expensive grade of diesel fuel provided by the fuel vendor or obtained by government testing). Epistemic uncertainty can be particularly problematic because it is a type of systematic uncertainty which cannot, for a given set of parameter values, be removed by increasing the number of runs of the simulation. For example, if the runs all use a fuel efficiency PDF mean for the less expensive grade of diesel that is too high, then all (or at least most) runs of the simulation for the less expensive grade of diesel will overestimate fleet fuel efficiency.

### BRIEF SUMMARY

In some aspects disclosed herein, a non-transitory storage medium stores: a plurality of event modules readable and executable by an electronic processor to define events that transform attributes of members of at least one population class in accord with probabilistic event models defined by event model parameter files; application programming interfaces (APIs) defining flow of members of the at least one population class amongst the event modules; and instructions readable and executable by the electronic processor to perform a discrete event simulation (DES) method. The DES method comprises: incrementing a timepoint for the DES until a stopping criterion is met; at increments of the timepoint, invoking event modules of the plurality of event modules to process members of the at least one population class in accord with flow of the members of the at least one population class amongst the event modules of the plurality of event modules as defined by the APIs; and responsive to the stopping criterion being met, outputting DES data comprising attributes of the members of the at least one population class at least at the time increment at which the stopping criterion is met.

In some aspects disclosed herein, a discrete event simulator is disclosed for performing DES. The discrete event simulator comprises an electronic processor and a non-transitory storage medium storing instructions readable and executable by the electronic processor to implement a population generator module, a plurality of event modules, application programming interfaces (APIs), and a DES controller. The population generator module is configured to, for each of at least one population class, process a population class parameters file defining probability density functions (PDFs) for attributes of the population class to generate members of the population class with attributes distributed over the generated members in accord with the PDFs for the attributes of the population class. The plurality of event modules define events that process members of the at least one population class to transform attributes of the members of the at least one population class in accord with probabilistic event models defined by event model parameter files. The APIs define flow of the members of the at least one population class amongst the event modules of the plurality of event modules. The DES controller includes a random number generator, and is configured to perform a DES including: invoking the population generator module to generate members of the at least one population class wherein the population generator module draws random numbers from the random number generator of the DES controller in generating the members of the at least one population class; incrementing a timepoint for the DES until a stopping criterion is met; and at increments of the timepoint, invoking event modules of the plurality of event modules to process members of the at least one population class in accord with flow of the members of the at least one population class amongst the event modules of the plurality of event modules as defined by the APIs, wherein the event modules draw random numbers from the random number generator of the DES controller in processing the members of the at least one population class.

In some aspects disclosed herein, a DES method is disclosed. Using an electronic processor, a timepoint for the DES is incremented until a stopping criterion is met. At increments of the timepoint, event modules of a plurality of event modules are executed using the electronic processor to process members of at least one population class in accord with flow of the members of the at least one population class amongst the event modules of the plurality of event modules as defined by a plurality of application programming interfaces (APIs). The event modules process the members of the at least one population class to transform attributes of the members of the at least one population class in accord with probabilistic event models defined by event model parameter files. Responsive to the stopping criterion being met, DES data are written to a non-transitory storage medium. The DES data comprises attributes of the members of the at least one population class at least at the time increment at which the stopping criterion is met.

In some aspects disclosed herein, a non-transitory storage medium stores instructions readable and executable by an electronic processor to perform a simulation method for simulating a system represented by a system model having parameters with associated probability density functions (PDFs). The simulation method comprises performing M outer loops where M is an integer greater than one. Each outer loop includes: for each parameter, randomly drawing a value for the parameter with replacement from a set of discrete sampling points distributed over the PDF associated with the parameter; and running N simulations of the system using the system model with the randomly drawn values for the parameters wherein N is an integer greater than one and simulation results for each simulation are stored in the non-transitory storage medium annotated by the randomly drawn values for the parameters.

In some aspects disclosed herein, a simulator includes an electronic processor, and a non-transitory storage medium storing instructions readable and executable by the electronic processor to perform a simulation method for simulating a system represented by a system model having parameters with associated probability density functions (PDFs). The simulation method comprises: selecting a set of discrete sampling points for each parameter of the system model including, for each parameter whose PDF is a non-uniform and non-multinomial distribution, selecting the set of discrete sampling points for the parameter as values for which the cumulative density function (CDF) corresponding to the PDF have percentile values belonging to a set of predefined percentile values; and performing M outer loops where M is an integer greater than one. Each outer loop includes: for each parameter, randomly drawing a value for the parameter with replacement from a set of discrete sampling points distributed over the PDF associated with the parameter; and running N simulations of the system using the system model with the randomly drawn values for the parameters wherein N is an integer greater than one and simulation results for each simulation are stored in the non-transitory storage medium annotated by the randomly drawn values for the parameters.

In some aspects disclosed herein, a simulation method is disclosed for simulating a system represented by a system model having parameters with associated probability density functions (PDFs). The simulation method comprises: selecting a set of discrete sampling points for each parameter of the system model including, for each parameter whose PDF is a non-uniform and non-multinomial distribution, selecting the set of discrete sampling points for the parameter to correspond to a set of predefined percentile values, wherein the parameter value corresponding to a given predefined percentile value PV represented as a decimal value is given by X satisfying the equation: PV=∫_{-∞}^{X} PDF(x)dx; and performing M outer loops where M is an integer greater than one. Each outer loop includes: for each parameter, randomly drawing a value for the parameter with replacement from a set of discrete sampling points distributed over the PDF associated with the parameter; and running N simulations of the system using the system model with the randomly drawn values for the parameters wherein N is an integer greater than one and simulation results for each simulation are stored in the non-transitory storage medium annotated by the randomly drawn values for the parameters.

In any of the above aspects, the method may further include computing a variance of expectations Var(E[Y|X]) which represents the variance due to uncertainty in the model's parameters, where X is the set of parameters of the system model, Y is the simulation results, and E stands for expectation. In any of the above aspects, the method may further include computing an expectation of variance E[Var(Y|X)] which represents the variance due to inherent stochasticity of the model, where X is the set of parameters of the system model, Y is the simulation results, and E stands for expectation.

### BRIEF DESCRIPTION OF THE DRAWINGS

The following is a brief description of the drawings, which are presented for the purposes of illustrating the exemplary embodiments disclosed herein and not for the purposes of limiting the same.

FIG. 1 diagrammatically shows a discrete event simulator.

FIG. 2 diagrammatically shows operation of the population generator module of the discrete event simulator of FIG. 1.

FIG. 3 diagrammatically shows the running of the DES by the discrete event simulator of FIG. 1.

FIG. 4 diagrammatically shows an overview of setting up and running a DES using the discrete event simulator of FIG. 1.

FIG. 5-12 diagrammatically illustrate an example of setting up and using the discrete event simulator of FIG. 1 for a nonlimiting illustrative example task of simulating medical services delivery and payment collection.

FIG. 13 diagrammatically shows a simulator system.

FIG. 14 diagrammatically shows operation of the simulator of FIG. 13 with dynamic Monte Carlo sampling of the of the simulator system of FIG. 13.

### DETAILED DESCRIPTION

A more complete understanding of the methods and apparatuses disclosed herein can be obtained by reference to the accompanying drawings. These figures are merely schematic representations and are not intended to indicate relative size and dimensions of the assemblies or components thereof.

Although specific terms are used in the following description for the sake of clarity, these terms are intended to refer only to the particular structure of the embodiments selected for illustration in the drawings, and are not intended to limit the scope of the disclosure. In the drawings and the following description below, it is to be understood that like numeric designations refer to components of like function.

In discrete event simulation (DES), various types of events can occur, with associated likelihoods usually represented by probability density functions (PDFs). A system state is defined by a set of state variables, which may change in response to (simulated) occurrences of events. The system state and the events are strongly interrelated in the DES. For example, in a DES run to simulate medical services delivery and payment collection, the system state is defined by the attributes of patients, doctors, medical institutions, medical insurance companies, and so forth. The various types of events that may occur include various medical diagnosis events, various medical treatment (e.g. surgery) events, various treatment outcome events, and so forth. Events generally change the system state in probabilistic ways, e.g. the treatment outcome event may output a state change of complete patient recovery, partial patient recovery, or no patient improvement, each with some defined PDF. Similarly, an insurance reimbursement claim decision event may output a state change of full payment, partial payment, or claim denial, again each with some defined PDF. The PDFs may themselves be functionally dependent on the system state, e.g. the likelihood of a partial payment may be higher if the system state includes the requested reimbursement amount being higher than the permitted reimbursement amount for a claim code associated with the reimbursement claim; while the probability of a denial may be higher if an invalid claim code is associated with the reimbursement claim. Occurrences of events also depend on prior occurrences of events, e.g. a hip injury diagnosis event may lead to a hip surgery event that in turn leads to a post-operative recovery event. Events can also impact one another concurrently in time, e.g. the hip surgery event may concurrently trigger a hip surgery reimbursement insurance claim event.

Due to these complex interrelationships between events, and between events and the system state, existing discrete event simulators typically have hard-coded event flows, with events represented by hard-coded event models with custom probability engines and implementing event models tailored to the specific DES to be performed.

However, it is recognized herein that this approach has disadvantages. For example, implementing changes to the assumptions driving the DES can be difficult due to the hardcoded links. Reuse of event models for different event types in different contexts is hindered. A further disadvantage is duplication of components, such as probability engines, amongst the various event models.

Discrete event simulator embodiments disclosed herein provide a DES controller that enables dynamic selection of event modules in use and links them together with an object-oriented design. Additionally, data import (to specify scenarios) and data export (for analysis of the system results, including those dynamically selected events) is facilitated by the modularization design which resolves of those issues and makes for rapid and easy development/integration of new events and for sensitivity testing. In tests, employing the DES simulator disclosed herein has reduced event implementation for the DES from a 16-20 hour process to a 5 hour process.

In illustrative embodiments, plain-text configuration of event modules provides for ad-hoc addition and subtraction of components from a given DES run. Integration specification provides for seamless addition and subtraction of new events. Dynamic object state facilitates recording for data export which captures the attributes of the full object and exports it, for example in JavaScript Object Notation (JSON) or another standard data interchange format. A given simulation can be implemented as a design specification and a set of Application Programming Interfaces (APIs) that are integrated together by the DES controller to enable efficient software development, testing, and experimentation. The plain-text configuration files are specified by a user and then passed into a command line function to generate exported data for analysis. This analysis is used to estimate the projected impact of a scenario change.

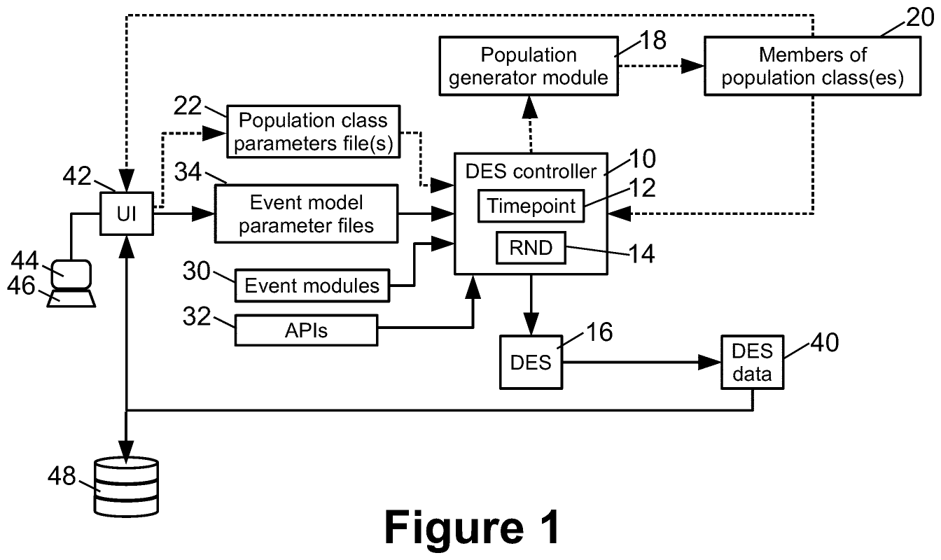

With reference to FIG. 1, a DES system is centered on a DES controller 10, which in the illustrative embodiment includes a single random number (RND) generator 14 and maintains a timepoint 12 for performing a DES 16. The DES controller 10 coordinates execution of a population generator module 18 that generates members 20 of one or more population classes defined by corresponding population class parameter files 22. In running the DES 16, the DES controller 10 increments the timepoint 12 for the DES 16 until a stopping criterion is met (e.g., a pre-defined timespan for the DES 16, or a stopping criterion based on the system state of the DES 16 as defined by the members 20 of the at least one population class). At increments of the timepoint 12, event modules 30 of the plurality of event modules are invoked to process the members 20 of the at least one population class in accord with flow of the members 20 of the at least one population class amongst the event modules of the plurality of event modules as defined by Application Programming Interfaces (APIs) 32. The event modules 30 define events that transform attributes of the members 20 of at least one population class in accord with probabilistic event models defined by event model parameter files 34. The DES 16 outputs DES data 40, which may for example comprise attributes of the members 20 of the at least one population class at least at the time increment at which the stopping criterion is met. Additionally or alternatively, the output DES data 40 may comprise attributes of the members 20 of the at least one population class at one or more time increments prior to the time increment at which the stopping criterion is met. A user interface (UI) 42 is provided for user interaction with the discrete event simulator. For example, the UI 42 may operate in conjunction with a display 44 (e.g. an LCD display, plasma display, OLED display, or so forth) and at least one user input device 46 (e.g. keyboard, mouse, trackball, touch-sensitive display, et cetera) operatively connected with an electronic processor (not shown) implementing the discrete event simulator. The UI 42 may, for example, provide user interfacing for user review of the members 20 of the at least one population class, and user review of output DES data 40. Furthermore, the DES data 40 may be written to a non-transitory storage medium 48, for example as one or more Javascript Object Notation (JSON) files.

As noted, the discrete event simulator is suitably implemented by an electronic processor of a computer or other electronic processing device. For example, the discrete event simulator may be implemented on a desktop computer, notebook computer, server computer, plurality of server computers (e.g., forming a server cluster, cloud computing resource, or so forth), various combinations thereof or so forth. In particular, the non-transitory storage medium 48 may store: (i) the plurality of event modules 30 which are software (i.e. instructions) readable and executable by the electronic processor to define events that transform attributes of members of at least one population class in accord with probabilistic event models defined by event model parameter files; (ii) the population generator module 18 which is software (i.e. instructions) readable and executable by the electronic processor to generate the members 20 of the at least one population class; (iii) the APIs 32 defining flow of members of the at least one population class amongst the event modules; and (iv) instructions readable and executable by the electronic processor to implement the DES controller 10 so as to perform the DES 16. It will be appreciated that the DES data 40 may be stored on the same non-transitory storage medium 48 (as shown), or a different non-transitory storage medium, than that which stores the event modules 30, population generator module 18, APIs 32, and DES controller 10. In general, the non-transitory storage medium or media 48 may be a hard disk, RAID, or other magnetic storage medium, a solid state drive (SSD) or other electronic storage medium, an optical disk or other optical storage medium, various combinations thereof, or so forth.

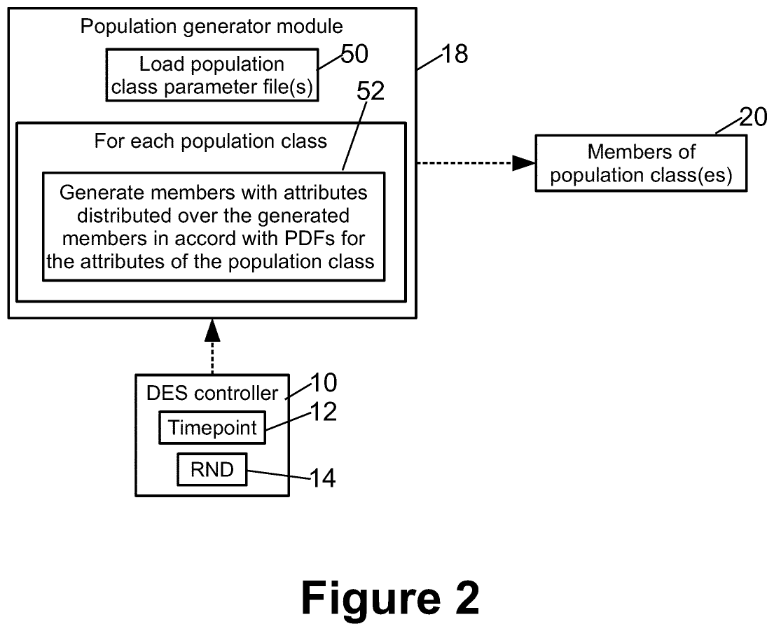

With reference to FIG. 2, operation of the population generator module 18 is described. At operation 50, the population class parameter file 22 for each population class is loaded. At operation 52, for each population class, members 20 are generated. In generating each member, the attributes are selected using the corresponding attribute PDFs defined in the parameter file 22, by drawing random numbers from the random number generator 14 of the DES controller 10. As a result, the members 22 of the at least one population class have attributes distributed over the generated members in accord with the PDFs the attributes of the population class.

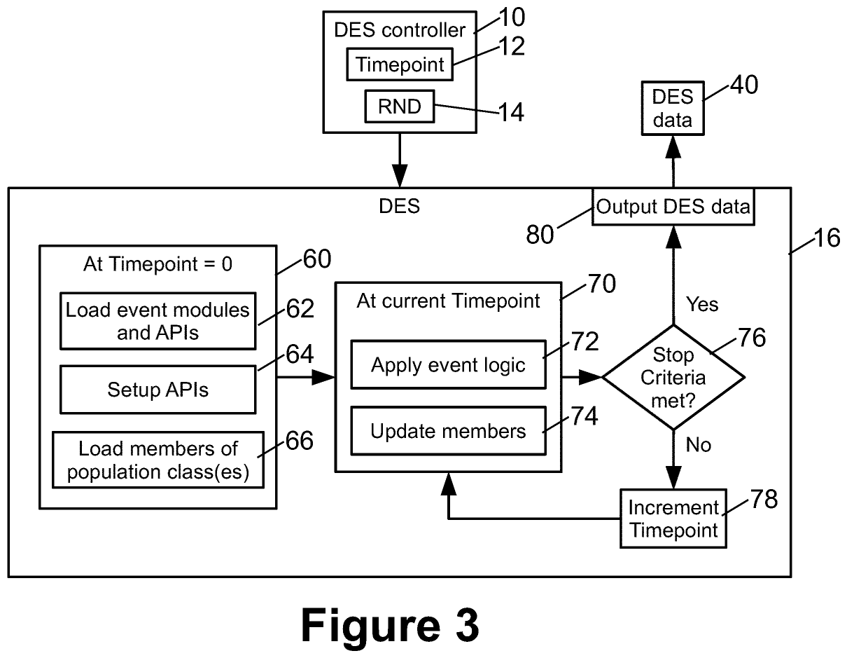

With reference to FIG. 3, the method of performing the DES 16 is described. The DES controller 10 initializes the DES 16 at initialization 60. This includes loading the event modules 30 and APIs 32 in an operation 62; setting up the APIs 32 in an operation 64; and loading the members 20 of the population class or classes in an operation 64 (which were previously generated by the population generator module 18 as described with reference to FIG. 2). After the initialization 60, the DES controller 10 increments the timepoint 12 and, at each (current) timepoint, performs an update process 70 which includes applying the event logic 72 and updating 74 the members 20 of the population class or classes. The operations 72, 74 entail invoking event modules 30 to process members 20 of the at least one population class in accord with flow of the members of the at least one population class amongst the event modules as defined by the APIs 32. When an event module 30 is invoked to process members of the population class(es), it transforms attributes of the members in accord with probabilistic event models defined by the event model parameter files 34. It should be noted that, depending upon the nature of the event being simulated, a single invocation of an event module 30 may process two or more members of the same population class and/or of different population classes. In the ongoing nonlimiting illustrative example of a DES run to simulate medical services delivery and payment collection, for example, a surgery event may operate on (i.e. process) a patient (i.e. a member of a patient class) and a surgeon (i.e., a member of a doctor class) and/or a hospital (i.e. a member of a medical institution class). The APIs 32 define flows between event modules within a timepoint or across successive timepoint increments, e.g. the APIs 32 may define that the patient processed by the surgery event then flows to a post-operative recovery event and to a surgery reimbursement claim request event.

After each timepoint is processed, at a decision operation 76 it is determined whether the stopping criterion is met. If not, then the timepoint 12 is incremented at operation 78 and flow passes back to the update process 70 for the next (now-current) timepoint. On the other hand, if at the decision 76 it is determined that the stopping criterion is met, then the DES 16 terminates and the DES data 40 is output at an output operation 80.

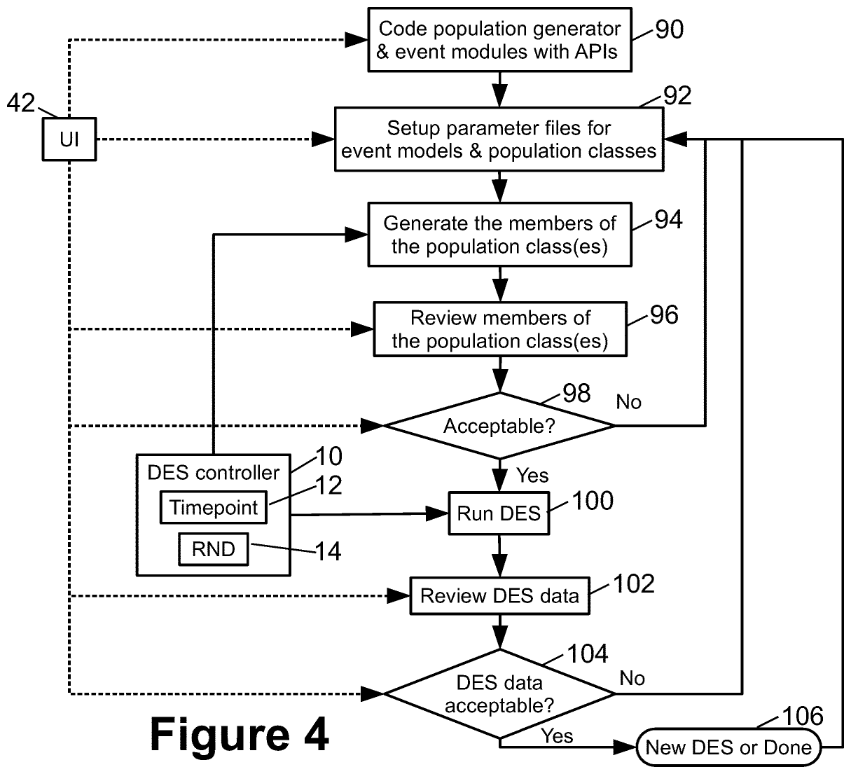

With reference to FIG. 4, an overview is presented of setting up and running a DES using the discrete event simulator of FIG. 1. At an operation 90, the population generator 18 and event modules 30 are coded, and the APIs 32 are set up. Preferably, the event modules 30 are object-oriented programming (OOP) objects. As such, they may be grouped into module groups. Each module group has a group-level class definition, and event modules belonging to a module group inherit the group-level class definitions of the module group to which the modules belong. Preferably, the population generator module 18 is also an OOP object, and optionally may also be coded as a plurality of OOP population generator modules that are grouped into one or more generator module groups. In this case, each generator module group has a group-level class definition, and population generator modules belonging to a generator module group inherit the generator group-level class definitions of the generator module group to which the population generator modules belong. At an operation 92, the parameter files for the event models and population classes are set up.

It will be appreciated that this OOP paradigm facilitates more rapid performance of the operations 90, 92. For example, in the ongoing nonlimiting illustrative example of a DES run to simulate medical services delivery and payment collection, a surgery module group can be coded, and various specific types of surgery can then be implemented using the inheritance property of OOP so that the surgery module for each specific type of surgery inherits the surgery group-level class definitions of the surgery module group (e.g. definitions of the diagnosis calling for the surgery, the claim code of the surgery, and so forth) with specific values for these definitions appropriate for the specific type of surgery being provided by the parameter files set up in the operation 92. Likewise, for simulating different populations of patients (e.g., a first patient population class corresponding to a first disease or medical condition, a second patient population class corresponding to a second disease or medical condition, et cetera), a patient generator module group can be coded which defines group-level class definitions such as demographic, gender, and age distributions, and each population generator module (e.g. for the first population class, the second population class, et cetera) inherits the patient group-level class definitions of the patient module group with specific values for these definitions appropriate for the specific type of population to be generated by the specific population generator module being provided by the parameter files set up in the operation 92.

Optionally, if the population generator 18 and/or one or more of the event modules 30 was previously coded for some earlier DES task, then it can be reused and not newly coded in the operation 90. Rather, in the operation 92 a suitable parameter file is set up to set the specific values for the inherited group-level class definitions. Similarly, if a module group to which an event module is closely related has already been coded, then in the operation 90 the event module can be constructed using the module group with suitable setup of the parameter file in the operation 92; likewise, a population module group can be leveraged to construct a specific population generator module falling within that population module group definition.

In an operation 94, the population generator module 18 is applied to generate the members 20 of the at least one population class, as already described with reference to FIG. 2. At an operation 96, the user can review the members 20 via the UI 42. This may entail reviewing individual members, and/or looking at the statistical profile of all generated members (or sub-groups thereof). At an operation 98 it is determined whether the generated members 20 are acceptable—if not, flow returns to operation 92 to adjust the parameters file defining the PDFs for the attributes of the members. (If needed, flow could also return to operation 90 to revise the underlying coding of the population generator module 18).

On the other hand, if at the operation 98 it is determined that the generated members 20 are acceptable, then at an operation 100 the DES 16 is run, as already described with reference to FIG. 3. At an operation 102, the output DES data 40 are reviewed by the user via the UI 42. This may entail reviewing DES data for individual members after being processed by the DES 16, and/or looking at the statistical profile of all generated members (or sub-groups thereof) after being processed by the DES 16. At an operation 104 it is determined whether the DES data 40 are acceptable—if not, flow returns to operation 92 to adjust the parameters file defining the PDFs for the event and/or population generator modules. (If needed, flow could also return to operation 90 to revise the underlying coding of the modules). On the other hand, if at the operation 104 it is determined that the DES data 40 are acceptable, then at an operation 106 simulation is complete. If a new DES is to be run, then flow passes back to operation 92 to adjust the parameter files to implement the new simulation.

It will be appreciated that the discrete event simulator described with reference to FIG. 1-4 can be used in simulating any system or process in which a members of a population undergo events that can be simulated. For example, the discrete event simulator described with reference to FIG. 1-4 can be employed to simulate traffic systems. In this case, the population classes may include a vehicle class (or a vehicle class group, with population classes such as trucks, cars, bicycles, et cetera inheriting the vehicle group-level class definitions), and/or drivers (or a driver class group, with population classes such as truck drivers, car drivers, bicycle riders, et cetera inheriting the driver group-level class definitions), and/or so forth. The event modules 34 may include event modules such as a cargo haul modules (or a cargo haul module group, with event modules such as long-haul, intra-city haul, et cetera inheriting the cargo haul module group-level class definitions), accident modules (or an accident module group, with event modules such as fender-bender, freeway accident, fatal accident, et cetera inheriting the accident module group-level class definitions), and/or so forth.

As another example, the discrete event simulator described with reference to FIG. 1-4 can be employed to simulate a computer network. In this case, the population classes may include an end-user class (or an end-user class group, with population classes such as home users, small business users, corporate users, et cetera inheriting the end-user group-level class definitions), and/or internet service providers (ISPs) (or an ISP class group, with population classes such as small, medium, or large ISPs inheriting the ISP class group definitions), and/or so forth. The event modules 34 may include event modules such as a video streaming modules (or a video streaming module group, with event modules for different bit rates or other granularities of video streaming inheriting the video streaming module group-level class definitions), audio streaming modules (or an audio streaming module group, with event modules for audio streaming at different bit rates inheriting the audio streaming group-level class definitions), and/or so forth.

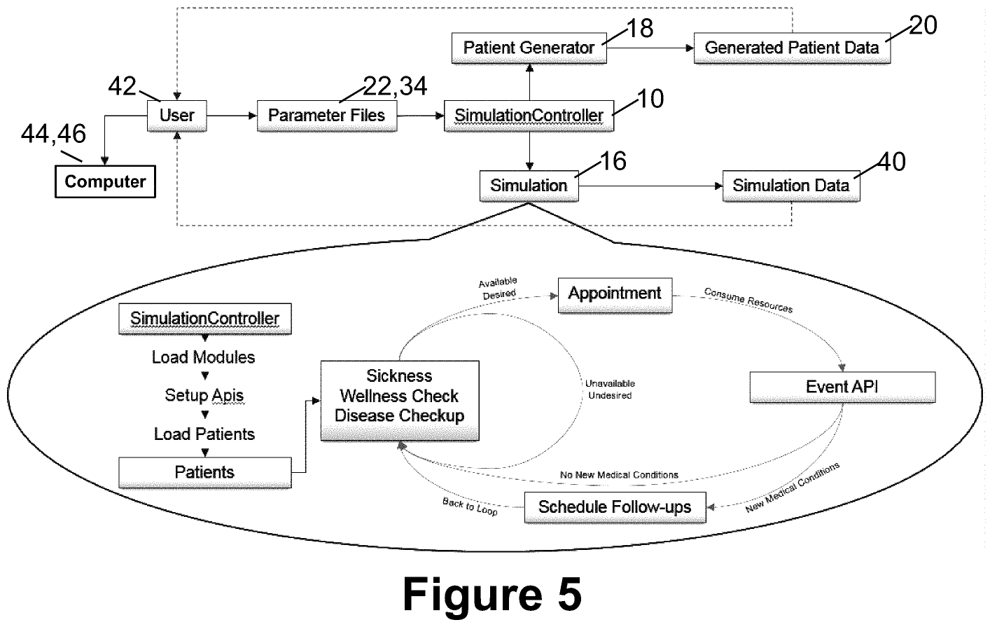

With reference to FIGS. 5-13, by way of nonlimiting further illustration, examples of using the discrete event simulator described with reference to FIG. 1-4 for simulating medical services delivery and payment collection is described. More specifically, the examples of FIGS. 5-13 pertain to simulations relating to a proposal in the United States to transform federally qualified health centers (FQHCs) into advanced primary care practices (APCPs), which is a demonstration project of the Centers for Medicare and Medicaid Services (CMS). The tasks included static simulation of Bundled Payments for Care Improvement (BPCI) Lower Extremity Joint Replacement. Employment of the discrete event simulator of FIGS. 1-4 in this task enables events to be specified in narrative form, with costs specified (to the care center and to CMS), with parameterized algorithms for events and costs. The use of the discrete event simulator of FIGS. 1-4 facilitated adding events, resources, medical conditions, or so forth as needed, provided flexibility with changing assumptions, and enabled rapid development of code for new modules.

With reference to FIG. 5, the discrete event simulator of FIG. 1 is shown in the upper portion, with components labeled as appropriate for the FQHC APCP task. The lower portion of FIG. 5 delineated by an oval corresponds to a task-specific embodiment of the processing depicted in FIG. 3.

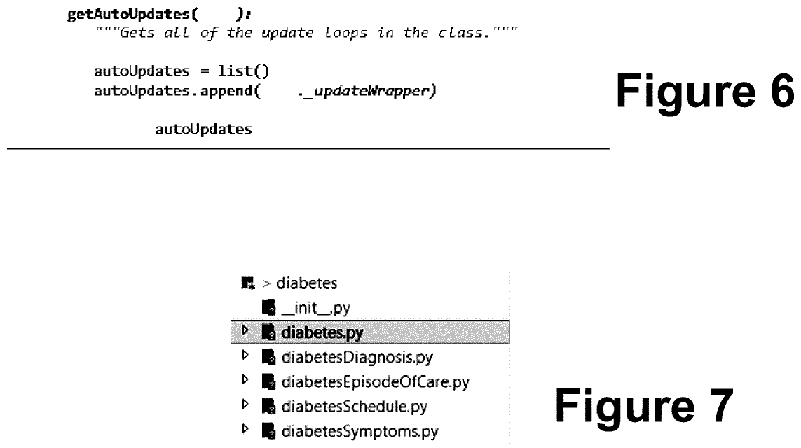

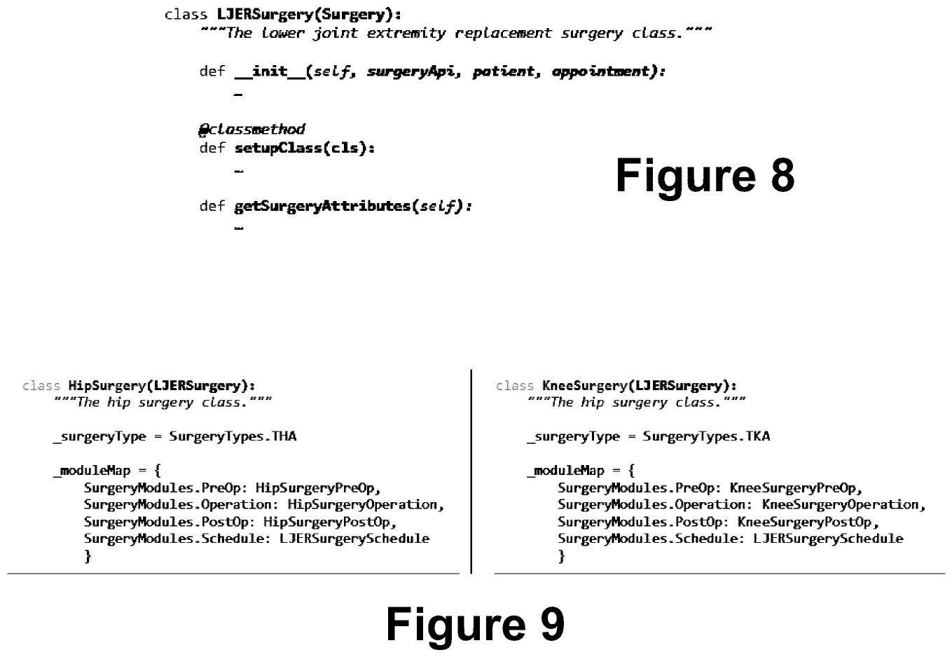

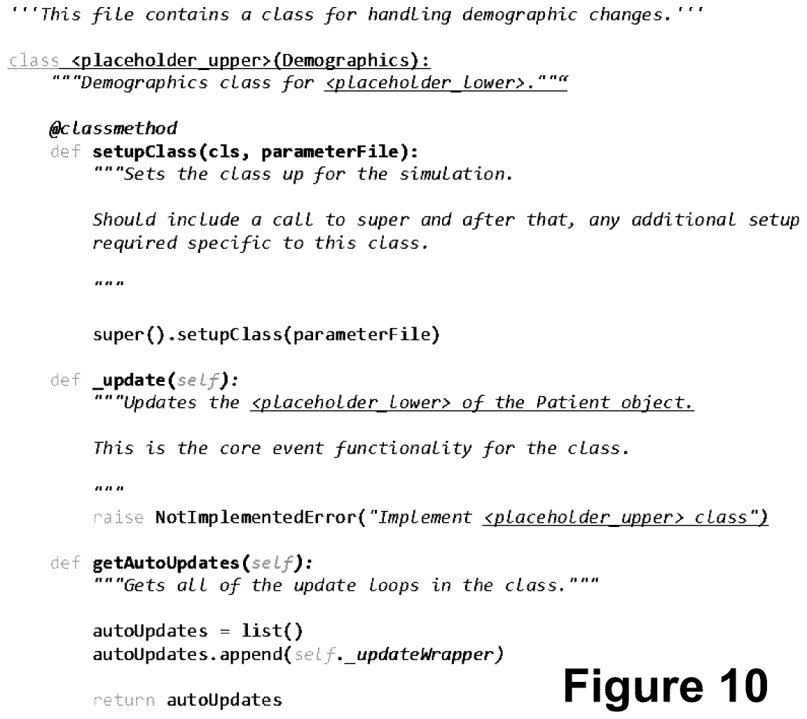

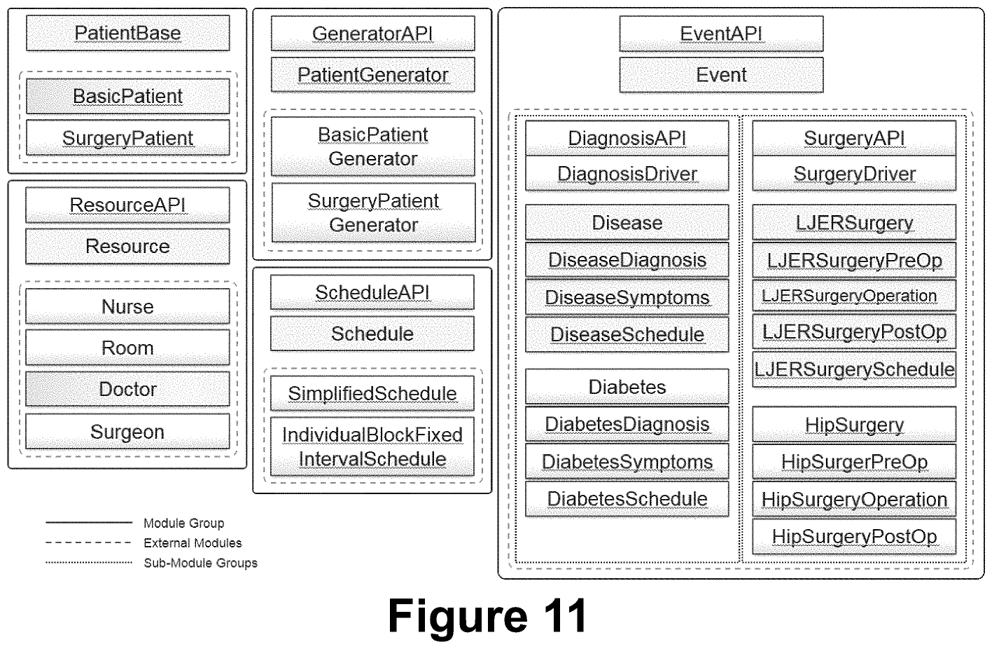

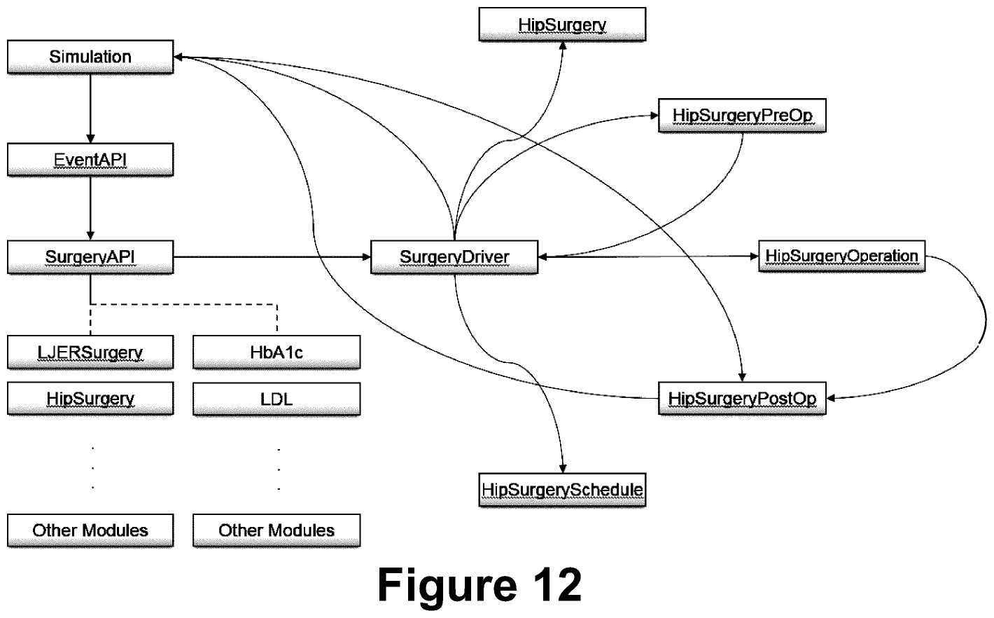

With reference to FIGS. 6-12, some illustrative embodiments of the event modules for the FQHC APCP task are described. The event modules for this task implement event logic for pre-operative tests, surgical operations, post-operative recovery, and various doctor-patient interactions. The illustrative discrete event simulator for the FQHC APCP task was coded in Python, and as shown in FIG. 6 leveraged some of python's leniency, e.g. utilizing code such as Assign method to variable and setattr( ). Event modules are object oriented programming (OOP) objects which may be grouped into module groups (i.e., super classes). The module groups have group-level class definitions, and event modules belonging to a module group inherit the group-level class definitions of the module group (i.e. super class) to which the event modules belong. Python also enables using methods to get additional, non-inherited attributes. FIG. 7 illustrates a module group “diabetes” with various event modules belonging to this group. FIGS. 8 and 9 illustrate another example, with FIG. 8 showing a module group (i.e. super class) for LJERSurgery (i.e., Lower Joint Extremity Replacement Surgery), and FIG. 9 showing two illustrative event modules (HipSurgery and KneeSurgery) belonging to the LJERSurgery module group. With reference to FIG. 10, a similar approach can be taken for templates for generating members of populations. Some modules may be constructed as multi-level hierarchical packages. FIG. 11 illustrates such a multi-level module hierarchical arrangement of templates for populations (patients, with lower-level templates for basic patient and surgery patient inheriting the attributes of the basic patient; and resources, with lower-level templates for nurse, room, doctor, and surgeon resources inheriting the attributes of the top-level resources template), and similar packages for event modules. Use of OOP objects for implementing the event modules breaks up large amount of code and increases reusability of code. FIG. 12 illustrates a portion of the flow of members (here patients) of the patient population class amongst the event modules, as defined by the APIs.

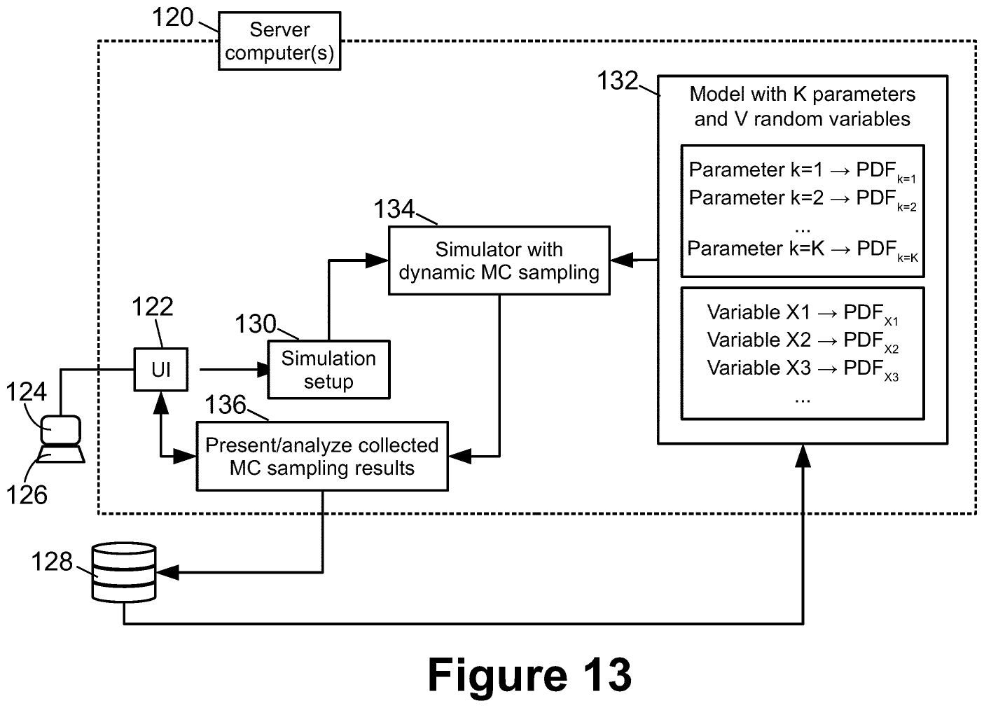

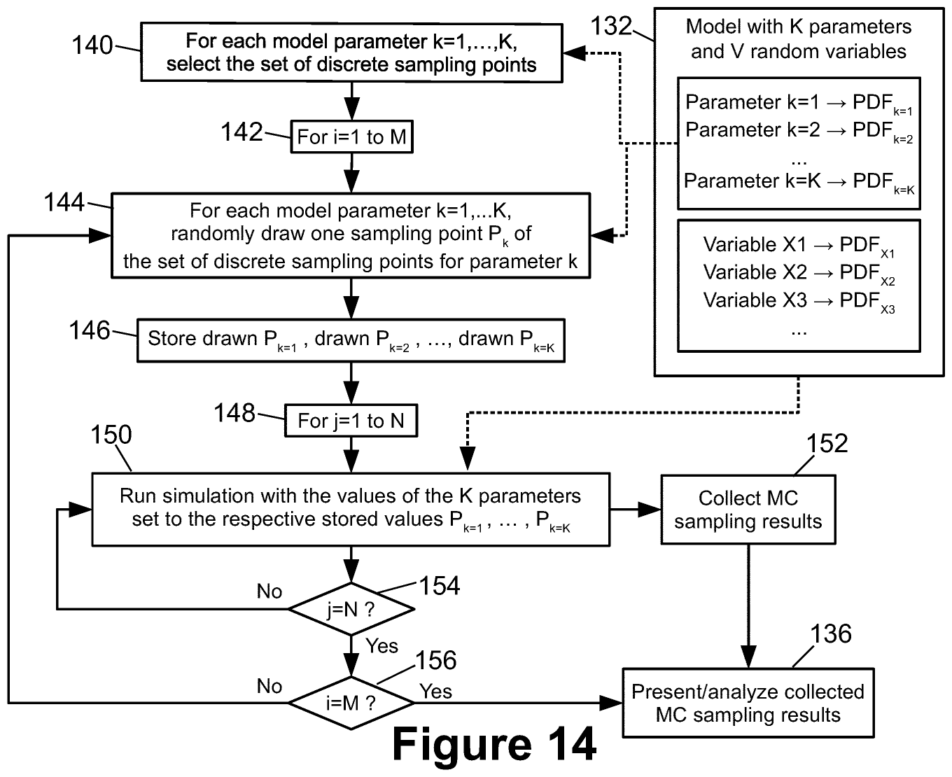

With reference to FIGS. 13 and 14, approaches are disclosed for addressing uncertainty in simulations such as those previously described.

In principle, epistemic uncertainty can be addressed by running the simulation with different sets of parameters in order to capture the variances of values of the model parameters. For a trucking fleet simulation as a non-limiting example, the prices of more expensive and less expensive grades of diesel can be represented by corresponding probability density functions (PDFs) capturing the expected pricing variation due to market forces (e.g., based on historical data for diesel pricing), and likewise the mean (and possibly variance) of the PDFs representing fuel efficiency of the respective grades of diesel can themselves be represented by PDFs (for example, based on variation in fuel efficiency amongst trucks in some test runs). Then, the models are re-run using the different sets of parameters a sufficient number of times to capture the statistical variation of the results due to the uncertainties in the pricing and fuel efficiency PDF mean parameters.

However, in practice, this approach is problematic with large, complex simulations, which can have dozens, hundreds, or thousands of parameters. For example, Latin hypercube sampling is an approach to uncertainty characterization requiring a set of parameters be fixed and simulated with multiple iterations, then incremented, and simulated with multiple iterations, resulting in a set of several combinations of ranges of parameters with both a within simulation uncertainty (from Monte-Carlo simulation effects), and uncertainty due to the input parameter. This approach typically requires an analyst to manually specify all combinations of parameters, and with a large number of parameters this quickly becomes a complicated and costly process to implement. For example, if there are 200 parameters and the Latin hypercube sampling analyzes 5 possible values for each parameter, then full analysis of the epistemic uncertainty would require analyzing 200!*5=4×10³⁷⁵ sets of parameters.

In approaches disclosed herein, Monte Carlo simulation is used to specify a set of parameter values drawn from a discrete set of possible parameter values, e.g. defined as a set of percentiles. This set of Monte Carlo-generated parameter values is used to perform a certain number of simulation iterations (e.g., user specified), and outputs information about both the values of the parameters (selected by the Monte Carlo sampling for the simulation) and the outputs (e.g., used to estimate variance). By the combination of using Monte Carlo sampling, and more specifically Monte Carlo sampling that draws from a discrete set of parameter values that are strategically chosen based on the PDF for parameter, e.g. by selecting designated percentile values, the disclosed simulator with Monte Carlo sampling can cover a representative number of sets of parameters without exhaustively processing the entire Latin hypercube. Moreover, it can advantageously do so in some embodiments with the user specifying only a few (e.g. three) command line arguments.

The disclosed approach of simulating with Monte Carlo sampling is described in further detail below.

Ideally, in order to quantify the uncertainty of the model, thousands of iterations should be done, varying the selection of input parameters such that a wide spread of their distributions were sampled. However, for a large, complex model with dozens, hundreds, or more parameters, there are significant computational limitations that constrain the number of iterations that can be run in a timely manner. However, reducing the number of parameter sets also constrains the variation in parameter selection. Since all calculations (e.g. expectation or variance) are done on only those model outputs (Y) that were generated by the same model input (X), it can be challenging to produce simulations with the same set of input parameter values with few iterations for a large number of input parameters.

In order to adequately sample the distribution of an input parameter without excessively increasing the number of iterations, the disclosed approach reduces the full distribution of each input parameter to a set of discrete sampling points, which are strategically chosen to span the distribution. The determination of the set of discrete sampling points can be dependent upon the distribution type of the PDF of the input parameter. For example, in the case of a parameter whose PDF is a non-uniform, non-multinomial distribution, the distribution in some embodiments is divided into six parts using percentiles (e.g. 10^{th}, 25^{th}, 50^{th}, 75^{th} and 90^{th}). The value of the distribution at a given percentile can be computed using the cumulative distribution function (CDF) of the PDF of the parameter. This is done by setting the CDF equal to the decimal value P of the percentile (for example, for the 25th percentile the CDF is set to 0.25; or, for the illustrative set of percentiles 10^{th}, 25^{th}, 50^{th}, 75^{th}, and 90^{th}, the corresponding decimal values are 0.1, 0.25, 0.5, 0.75, and 0.9) and solving. More particularly, given a PDF for a parameter, the corresponding CDF is given by:

CDF(X)=∫_{-∞}^{X}PDF(x)dx (1)

So, for example, to find the 25^{th} percentile sampling point, in Equation (1) CDF is set equal to the decimal value 0.25, and the resulting equation is solved for X, that is, solve the equation:

0.25=∫_{-∞}^{X}PDF(x)dx (2)

for the value of X. More generally, the sampling point X for a predefined percentile value is given by:

PV=∫_{-∞}^{X}PDF(x)dx (3)

where PV is the predefined percentile value represented as a decimal value.

As discussed elsewhere herein, the number of runs of the model simulation to generate sufficient variability statistics can be suitably chosen based on the largest set of discrete sampling points of any parameter. This tends to bias toward selecting the set of discrete sampling points to be as small as possible. On the other hand, if the set of discrete sampling points is too small then the variability of the parameter is not well sampled. In some illustrative embodiments, the set of discrete sampling points preferably has 10 sampling points or less. In some illustrative embodiments, the set of discrete sampling points preferably has 7 sampling points or less. In the illustrative example, the set of discrete sampling points in the case of a parameter whose PDF is a non-uniform, non-multinomial distribution has five sampling points, corresponding to the predefined percentiles 10^{th}, 25^{th}, 50^{th}, 75^{th}, and 90^{th} and determined by solving Equation (3) for X with PV set to the decimal value of a given percentile of the set of predefined percentiles. This is merely an illustrative example, and other sets of predefined percentiles are contemplated for use in this selection.

In the case of a parameter whose PDF is a uniform distribution over an interval, in some embodiments the set of discrete sampling points is chosen by dividing the interval of the uniform distribution into 6 equal pieces, thus defining a set of 5 discrete sampling points. (More generally, the interval of the uniform distribution can be divided into D+1 equal pieces, thus defining a set of D discrete sampling points).

In the case of a parameter whose PDF is a multinomial distribution (where “multinomial distribution” as used herein encompasses the limiting case of a binomial distribution), the parameter is restricted to discrete values belonging to a discrete set of possible values. For this type of parameter, various approaches can be taken. If the set of possible values is sufficiently small, then the set of discrete sampling points can be equal to the set of possible values of the multinomial distribution. If this would result in too many discrete sampling points, then the set of discrete sampling points can be a subset of the set of possible values of the multinomial distribution with the highest probability of occurrence in the multinomial distribution. An automated example of such an approach is to take the smaller of (i) the smallest set of values whose summed probability of occurrence in the multinomial distribution is at least some threshold value (e.g. at least 50%), or (ii) the set of five values whose individual probability of occurrence in the multinomial distribution is highest.

In the limiting case for a parameter whose PDF is a multinomial distribution, the set of discrete sampling points for a parameter having a multinomial distribution can be a single sampling point chosen as the value with the highest probability of occurrence in the multinomial distribution. This lattermost approach has certain advantages in certain cases: although it reduces accuracy of the quantification of model uncertainty, if there are only a few parameters whose PDFs are multinomial distributions then the impact of selecting only the single most probable value is usually acceptable, and this choice reduces computational complexity.

Still further, if a parameter is not part of the variability analysis, it will have a fixed value, with no PDF being associated with the parameter. (Alternatively, the fixed parameter could be viewed as having a PDF which is a delta distribution centered at the fixed value of the fixed parameter). In this case, the set of discrete sampling points for the fixed parameter is a single sampling point set to the fixed value of the parameter.

The simulator with dynamic Monte Carlo sampling runs in the following way. In the following, it is to be understood that random selection as used herein encompasses pseudorandom selection as that term is commonly used in the computer arts, e.g. using a seeded deterministic pseudorandom number generator. Furthermore, while the phrase “set of discrete sampling points” is used herein for simplicity, it is to be understood that for some parameters, such as a parameter whose PDF is a multinomial distribution, the “set of discrete sampling points” may include only a single discrete sampling point.

In each iteration of an outer loop, a value for each parameter is randomly drawn from its allowable set of discrete sampling points (e.g., generated from the set of 10^{th}, 25^{th}, 50^{th}, 75^{th}, and 90^{th} percentile points in some embodiments). In an inner loop, the simulation is run for N iterations using the same drawn parameter values. Once the N iterations of the inner loop are complete, the next iteration of the outer loop is performed, in which new values for the input parameters are drawn from their respective allowable sets of discrete sampling points, and the simulation is run for N iterations using the drawn parameter values. This is repeated for M iterations of the outer loop.

In the foregoing process, in the second and later iterations of the outer loop the random draw of parameter values is done with replacement, so that a parameter can take the same value more than once in successive iterations of the outer loop.

It will be recognized that the total number of simulations executed in this process is N×M. For the outer loop, M is preferably at least as large as the largest allowable set of discrete sampling points for any parameter, which allows for the possibility that an input parameter can take all possible values from their allowable set (although due to stochasticity this is not guaranteed to occur). If this minimum value for M is employed, then for a given value of N, the larger the largest allowable set of discrete sampling points for any parameter, the more model iterations (N×M) are needed. For large simulations, the goal of having a large enough allowable set to accurately characterize an input parameter's distribution is preferably balanced against the goal of running the simulations in a timely manner. In one nonlimiting illustrative example in which the largest set of discrete sampling points of any parameter is five points, the inner and outer loops are chosen to have N=30 and M=10, respectively, which leads to 30×10=300 simulation runs.

With reference now to FIG. 13, a simulator system employing the above-described principles is presented. The simulator system includes a server computer or other electronic processing device or devices 120 that runs the simulations, and a user interface (UI) 122 implemented by the electronic processor 120 in conjunction with a display 124 (e.g., an LCD display, plasma display, OLED display, or the like) and at least one user input device 126 (e.g. a keyboard, mouse, trackball, touch-sensitive display, various combinations thereof, and/or so forth). A non-transitory storage medium 128 stores instructions readable and executable by the electronic processor 120 to perform a simulation with dynamic Monte Carlo sampling as disclosed herein. While the illustrative electronic processor 120 is indicated in FIG. 13 as a server computer or computers, other embodiments are contemplated. For example, the electronic processor 120 may be implemented on a desktop computer, notebook computer, server computer, plurality of server computers (e.g., forming a server cluster, cloud computing resource, or so forth), various combinations thereof or so forth. The non-transitory storage medium 128 stores instructions readable and executable by the electronic processor 120 to implement the simulation system, and may also store various data generated by the simulations and/or by statistical analyses of the data generated by the simulations. It will be appreciated that the simulation and/or analysis data may be stored on the same non-transitory storage medium 128 (as shown in FIG. 13), or a different non-transitory storage medium, than that which stores the simulation software. In general, the non-transitory storage medium 128 may include a hard disk, RAID, or other magnetic storage medium, a solid state drive (SSD) or other electronic storage medium, an optical disk or other optical storage medium, various combinations thereof, or so forth.

With continuing reference to FIG. 13, a user (e.g. analyst) operates the UI 122 to perform simulation setup 130. This may entail operations such as loading a commercial discrete event simulation (DES) or other probabilistic simulation software package (e.g. AnyLogic simulation tool available from AnyLogic; MathWorks Simulation Software available from MathWorks Inc., FlexSim available from FlexSim Software Products Inc., or so forth) onto the computer 120, and constructing a system model 132 of the system to be simulated in accord with the syntax and formatting of the chosen commercial simulation software package. Alternatively, DES or other probabilistic simulation software can be custom written, and the system model 132 of the system to be simulated constructed in accord with the syntax and formatting required by the custom simulation software. As indicated in FIG. 13, without loss of generality the system model 132 is assumed to include K parameters denoted as, k₁, . . . , k_{K} and a set of random variables denoted as X₁, X₂, X₃, . . . . As previously described, the K parameters may include system-level parameters (e.g. the number of trucks in the trucking fleet in the ongoing example of simulation of a trucking fleet) as well as parameters for other aspects, such as parameters defining the random variables X₁, X₂, X₃, . . . . Parameters defining random variables might, for example, include the mean and variance parameters of a Gaussian PDF of a random variable for the fuel economies of the trucks of the fleet. In general, K>1, and more typically K may be a value in the tens or hundreds or higher.

Typically, the model 132 is stored on the non-transitory storage medium 128, and may be retrieved therefrom by the electronic processor 120 for simulating the system. The electronic processor 120 is further programmed to implement a simulator 134 with dynamic Monte Carlo (MC) sampling as disclosed herein. Results of the simulations are presented via the UI 122 in an operation 136, and the operation 136 may also include performing various statistical analyses on the data generated by the simulator 134.

With reference now to FIG. 14, operation of the simulator 134 with dynamic MC sampling is described. In an operation 140, the set of discrete sampling points is selected for each parameter k₁, . . . , k_{K}. This is done as previously described, e.g. for a parameter whose PDF is a non-uniform, non-multinomial distribution, the set of discrete sampling points may be selected percentiles, e.g. the 10^{th}, 25^{th}, 50^{th}, 75^{th} and 90^{th} percentiles, of the CDF corresponding to the PDF, computed by solving Equation (2) for X with the value of the CDF set to the decimal value of the percentile, as previously described. For a parameter whose PDF is a uniform distribution over an interval, a set of D discrete sampling points is suitably chosen to divide the interval of the uniform distribution into D+1 equal pieces. In the case of a multinomial distribution, the set of discrete sampling points may suitably consist of a single discrete sampling point corresponding to the value with highest probability of occurrence in the multinominal distribution. Alternatively, the set of discrete sampling points may consist of two discrete sampling points with the two highest probabilities of occurrence in the multinominal distribution; or so forth.

It will be recognized that the above-described approaches for implementing the operation 140 to select the set of discrete sampling points for each parameter k₁, . . . , k_{K} advantageously can be implemented automatically, without user input except possibly for user selection of the number of discrete sampling points for each parameter of a given PDF type (alternatively, this can be hard-coded). However, it is contemplated to have the user select the set of discrete sampling points for parameters deemed to be more likely to have an impact on model variability, and/or to select a higher number of discrete sampling points for (possibly user-identified) parameters deemed to be more likely to have an impact on model variability.

At operation 142, the outer loop begins, which (without loss of generality) is denoted by index i running from 1 to M, where M is an integer greater than one. At operation 144, for each model parameter k=1, . . . , K, one sampling point Pₖ of the set of sampling points for that parameter k is drawn. For example, if the set of sampling points for parameter k is the 10^{th}, 25^{th}, 50^{th}, 75^{th} and 90^{th} percentiles, then one of these values is randomly drawn. In an operation 146, the drawn parameter values P₁, . . . , P_{K} for the respective K parameters is stored (e.g. in the non-transitory storage medium 128, or in a random access memory (RAM) or other volatile memory of the electronic processing device 120).

At operation 148, the inner loop begins, which (without loss of generality) is denoted by index j running from 1 to N, where N is an integer greater than one. At operation 150, the simulation is run with the values of the K parameters set to respective values P₁, . . . , P_{K} which were drawn in the operation 144 and stored in the operation 146. The operation 150 simulates the system to be simulated as modeled by the model 132 with the stored values P₁, . . . , P_{K} for the respective K parameters. The simulation 150 is suitably performed by the commercial or custom DES or other probabilistic simulation software. At an operation 152, simulation data generated by the simulation 150 is collected, annotated with the stored values P₁, . . . , P_{K} for the respective K parameters. At loopback decision 154 for the inner loop, it is determined whether the inner loop has run N times—if not, then flow passes back to simulation operation 150 to perform a next run of the simulation, still using the same stored values P₁, . . . , P_{K} for the respective K parameters.

Once the inner loop 148, 150, 154 has run N iterations of the simulation 150 using the same stored values P₁, . . . , P_{K} for the respective K parameters, flow passes from loopback decision 154 for the inner loop to loopback decision 156 for the outer loop. At loopback decision 156 for the outer loop, it is determined whether the outer loop has run M times—if not, then flow passes back to the operation 144 where, for each model parameter k=1, . . . , K, a new sampling point Pₖ of the set of sampling points for that parameter k is drawn. This is done with replacement—hence, for any given parameter, it is possible that the same sampling point Pₖ may be drawn in successive iterations of the outer loop. In the operation 146, the parameter values P₁, . . . , P_{K} for this iteration of the outer loop are stored, and the inner loop 148, 150, 154 then runs M iterations of the simulation 150 using the values P₁, . . . , P_{K} for the respective K parameters drawn for this iteration of the outer loop.

Once M iterations of the outer loop have run, the full set of simulation data for the N×M runs of the simulation have been collected at collection operation 152, with the simulation data for each run annotated with the stored values P₁, . . . , P_{K} for the respective K parameters employed in that simulation run. Flow then passes from loopback decision 156 for the outer loop to operation 136 at which results of the simulations are presented via the UI 122, and various statistical analyses on the simulation data may be performed.

By way of nonlimiting example, a suitable statistical analysis of the simulation data suitably performed at the operation 136 is described. The illustrative approach uses the law of total variance to estimate the uncertainty in model output given fixed values of the model parameters. The law of total variance is given below:

Var(Y)=E[Var(Y|X)]+Var(E[Y|X]) (4)

and states that the total variance in the model output (Y) (i.e., the simulation results) can be decomposed into the sum of variance due to inherent stochasticity of the model E[Var(Y|X)], where E stands for expectation, Var stands for variance, and X is the set of model parameters; and variance due to uncertainty in the values of the parameters, (Var(E[Y|X])). The variance term E[Var(Y|X)] due to inherent stochasticity of the model corresponds to the aleatoric uncertainty, while the variance term Var(E[Y|X]) due to uncertainty in the values of the parameters corresponds to the epistemic uncertainty. Decomposing the total model variance in this way thus facilitates an understanding of the source of the uncertainty and provides information as to how to reduce it. If the uncertainty due to inherent stochasticity of the model (E[Var(Y|X)]) is a higher proportion of the total model uncertainty (Var(Y)), then uncertainty could be reduced by performing more model iterations. On the other hand, if uncertainty due to parameter input variability (Var(E[Y|X])) is a higher proportion of the total model output uncertainty (Var(Y)) then having more informed PDFs for the parameter distributions (for example, based on better data on the variability of diesel fuel prices, to take one possibility from the ongoing example of simulation of a trucking fleet) would be a better strategy to reducing model uncertainty than simply increasing the number of model iterations, as uncertainty may be as the result of the quality of evidence or insufficient data.

In the foregoing analysis, the variance is highly affected by the magnitude of the data values. Data scaled by a constant greater than one will have larger variance when compared to the unscaled version. Thus, the variance is not the best statistic for the purposes of comparing uncertainty between different model outputs. To address these concerns, the measure of uncertainty may additionally or alternatively be provided in terms of the coefficient of variation, denoted as CV(Y), which is the ratio between the standard deviation (or the square root of the variance) of the model output Y and the mean of the model output Y. The representation of the uncertainty in terms of this unit-less quantity may allow for more accurate comparisons between models or model outputs.

While the ongoing example in this description has been the example of simulation of the impact of grade of diesel fuel on a trucking fleet, it will be appreciated that the disclosed simulator with dynamic MC sampling of FIGS. 13 and 14 can be employed in conjunction with simulation of substantially any type of system in which the simulation employs a parameterized probabilistic model. By way of a few nonlimiting illustrative examples, the disclosed simulator with dynamic MC sampling can be employed in conjunction with simulation of roadway traffic systems (e.g. roadway networks, trucking fleets), aviation networks (e.g., airports, airline hub networks), computer networks (e.g. modeling data flow to study possible bottlenecks or the like), manufacturing facilities (e.g. modeling assembly lines), medical services (e.g. services delivery and reimbursement), financial systems (e.g. stock markets), and so forth.

In some embodiments, the simulator 134 of FIG. 13 may comprise the discrete event simulation (DES) of FIG. 1 in which various types of events can occur, and with associated likelihoods being represented by PDFs of the model 132. In this illustrative example, the event modules 30 define events that transform attributes of members of at least one population class in accord with probabilistic event models defined by event model parameter files. The APIs 32 define flow of members of the at least one population class amongst the event modules. The DES method includes: incrementing the timepoint 12 for the DES until a stopping criterion is met; at increments of the timepoint 12, invoking event modules to process members of the at least one population class in accord with flow of the members of the at least one population class amongst the event modules of the plurality of event modules as defined by the APIs; and responsive to the stopping criterion being met, outputting DES data comprising attributes of the members of the at least one population class at least at the time increment at which the stopping criterion is met. To incorporate into the simulator system of FIG. 13, the DES method suitably simulates a system represented by a system model having parameters with associated PDFs, and M outer loops are performed (where M is an integer greater than one). Each outer loop includes: for each parameter of the system model, randomly drawing a value for the parameter with replacement from a set of discrete sampling points distributed over the PDF associated with the parameter; and running the DES method as describe with reference to FIG. 1 for N times (where N is an integer greater than one) using the system model with the randomly drawn values for the parameters of the system model. The output DES data are annotated by the randomly drawn values for the parameters. Various statistical analyses can then be performed, such as computing the variance Var(E[Y|X]) due to uncertainty in the values of the parameters, and/or computing an expectation E[Var(Y|X)] representing variance due to inherent stochasticity of the system model.

The preferred embodiments have been illustrated and described. Obviously, modifications and alterations will occur to others upon reading and understanding the preceding detailed description. It is intended that the invention be construed as including all such modifications and alterations insofar as they come within the scope of the appended claims or the equivalents thereof.

1. A non-transitory storage medium storing:
a plurality of event modules readable and executable by an electronic processor to define events that transform attributes of members of at least one population class in accord with probabilistic event models defined by event model parameter files;
application programming interfaces (APIs) defining flow of members of the at least one population class amongst the event modules; and
instructions readable and executable by the electronic processor to perform a discrete event simulation (DES) method comprising:
incrementing a timepoint for the DES until a stopping criterion is met;
at increments of the timepoint, invoking event modules of the plurality of event modules to process members of the at least one population class in accord with flow of the members of the at least one population class amongst the event modules of the plurality of event modules as defined by the APIs; and
responsive to the stopping criterion being met, outputting DES data comprising attributes of the members of the at least one population class at least at the time increment at which the stopping criterion is met.

2. The non-transitory storage medium of claim 1 wherein:
the DES method simulates a system represented by a system model having parameters with associated probability density functions (PDFs); and
the non-transitory storage medium further stores instructions readable and executable by the electronic processor to:
perform M outer loops where M is an integer greater than one, each outer loop including:
for each parameter of the system model, randomly drawing a value for the parameter with replacement from a set of discrete sampling points distributed over the PDF associated with the parameter; and
running the DES method N times using the system model with the randomly drawn values for the parameters of the system model wherein N is an integer greater than one, wherein the output DES data are annotated by the randomly drawn values for the parameters.

3. The non-transitory storage medium of claim 1 further storing:
a population generator module readable and executable by the electronic processor to generate the members of the at least one population class by, for each population class, processing a population class parameters file defining probability density functions (PDFs) for attributes of the population class to generate members of the population class with attributes distributed over the generated members in accord with the PDFs for the attributes of the population class.

4. The non-transitory storage medium of claim 1 further storing a single random number generator, wherein the population generator module draws random numbers from the single random number generator in generating the members of the at least one population class and the event modules draw random numbers from the single random number generator in processing the members of the at least one population class.

5. The non-transitory storage medium of claim 1 further storing instructions readable and executable by the electronic processor to implement a user interface (UI) operating in conjunction with a display and at least one user input device operatively connected with the electronic processor for:
user review of the members of the at least one population class; and
user review of output DES data.

6. The non-transitory storage medium of claim 1 wherein the output DES data further comprises attributes of the members of the at least one population class at one or more time increments prior to the time increment at which the stopping criterion is met.

7. The non-transitory storage medium of claim 1 wherein the event modules of the plurality of event modules comprise object-oriented programming (OOP) objects grouped into module groups, wherein the module groups have group-level class definitions and event modules belonging to a module group inherit the group-level class definitions of the module group to which the event modules belong.

8. The non-transitory storage medium of claim 7 wherein:
the at least one population class includes at least a patients population class; and
the plurality of event modules includes one or more medical diagnosis modules belonging to a medical diagnosis module group, one or more medical surgery event modules belonging to a medical surgery module group, and one or more medical insurance reimbursement event modules belonging to a medical insurance reimbursement module group.

9. A discrete event simulator for performing DES, the discrete event simulator comprising:
an electronic processor; and
a non-transitory storage medium as set forth in claim 1 wherein the instructions stored on the non-transitory storage medium are readable and executable by the electronic processor.

10. A discrete event simulation (DES) method comprising:
executing a population generator module using an electronic processor to generate members of at least one population class by, for each population class, processing a population class parameters file defining probability density functions (PDFs) for attributes of the population class to generate the members of the population class with attributes distributed over the generated members of the population class in accord with the PDFs for the attributes of the population class;
using the electronic processor, incrementing a timepoint for the DES until a stopping criterion is met;
at increments of the timepoint, executing event modules of a plurality of event modules using the electronic processor to process the members of the at least one population class in accord with flow of the members of the at least one population class amongst the event modules of the plurality of event modules as defined by a plurality of application programming interfaces (APIs), wherein the event modules process the members of the at least one population class to transform attributes of the members of the at least one population class in accord with probabilistic event models defined by event model parameter files; and
responsive to the stopping criterion being met, writing DES data to a non-transitory storage medium wherein the DES data comprises attributes of the members of the at least one population class at least at the time increment at which the stopping criterion is met.

11. The DES method of claim 10 further comprising:
providing a user interface (UI) implemented by the electronic processor and a display and at least one user input device operatively connected with the electronic processor, the UI providing for user review of the members of the at least one population class and for user review of the DES data on the display.

12. A non-transitory storage medium storing instructions readable and executable by an electronic processor to perform a simulation method for simulating a system represented by a system model having parameters with associated probability density functions (PDFs), the simulation method comprising:
performing M outer loops where M is an integer greater than one, each outer loop including:
for each parameter, randomly drawing a value for the parameter with replacement from a set of discrete sampling points distributed over the PDF associated with the parameter; and
running N simulations of the system using the system model with the randomly drawn values for the parameters wherein N is an integer greater than one and simulation results for each simulation are stored in the non-transitory storage medium annotated by the randomly drawn values for the parameters.

13. The non-transitory storage medium of claim 12 wherein the simulation method further comprises:
for each parameter whose PDF is a non-uniform, non-multinomial distribution, selecting the set of discrete sampling points for the parameter as values for which the cumulative density function (CDF) corresponding to the PDF have percentile values belonging to a set of predefined percentile values.

14. The non-transitory storage medium of claim 13 wherein the set of predefined percentile values consists of 10 or fewer predefined percentile values.

15. The non-transitory storage medium of claim 12 wherein the simulation method further comprises:
for each parameter whose PDF is a non-uniform, non-multinomial distribution, selecting the set of discrete sampling points for the parameter to correspond to a set of predefined percentile values wherein the parameter value corresponding to a given predefined percentile value PV represented as a decimal value is given by X satisfying the equation: PV=∫_{-∞}^{X}PDF(x)dx.

16. The non-transitory storage medium of claim 12 wherein the simulation method further comprises:
for each parameter whose PDF is a uniform distribution over an interval, selecting the set of discrete sampling points for the parameter the set of D discrete sampling points that divide the uniform distribution into D+1 equal pieces.

17. The non-transitory storage medium of claim 12 wherein the simulation method further comprises:
for each parameter whose PDF is a multinomial distribution, selecting the set of discrete sampling points for the parameter as the single value with highest probability of occurrence in the multinomial distribution.

18. The non-transitory storage medium of claim 12 wherein the non-transitory storage medium further stores instructions readable and executable by the electronic processor to perform a statistical analysis on the simulation results including computing a variance Var(E[Y|X]) due to uncertainty in the values of the parameters, where X is the set of parameters of the system model, Y is the simulation results, and E stands for expectation.

19. The non-transitory storage medium of claim 12 wherein the non-transitory storage medium further stores instructions readable and executable by the electronic processor to perform a statistical analysis on the simulation results including computing an expectation E[Var(Y|X)] representing variance due to inherent stochasticity of the system model, where X is the set of parameters of the system model, Y is the simulation results, and E stands for expectation.

20. A simulator comprising:
an electronic processor; and
a non-transitory storage medium as set forth in claim 12 wherein the instructions stored on the non-transitory storage medium are readable and executable by the electronic processor.
